(19) United States
(12) Patent Application Publication
(10) Pub. No.: US 2025/0185923 A1
(43) Pub. Date: Jun. 12, 2025

(54) SPHYGMOMANOMETER

(71) Applicant: OMRON HEALTHCARE Co., Ltd., Kyoto (JP)

(72) Inventors: Tatsunori ITO, Kyoto (JP); Hiroyuki KANDA, Kyoto (JP); Yuta KUDO, Kyoto (JP)

(21) Appl. No.: 19/058,801

(22) Filed: Feb. 20, 2025

Related U.S. Application Data
(63) Continuation of application No. PCT/JP2023/028436, filed on Aug. 3, 2023.

(30) Foreign Application Priority Data
Dec. 9, 2022 (JP) 2022-197343

(51) Int. Cl. A61B 5/0205; A61B 5/00; A61B 5/021; A61B 5/022; A61B 5/024

(52) U.S. Cl. CPC A61B 5/0205; A61B 5/02108; A61B 5/02225; A61B 5/024; A61B 5/7264; A61B 5/7282; A61B 2560/0462; A61B 2562/0247

(57) ABSTRACT

A blood pressure measurement unit configured to measure blood pressure of a user based on a pulse wave signal superimposed on a cuff pressure signal detected when increasing or decreasing cuff pressure that indicates internal pressure of a cuff attached to the user; a pulse rate measurement unit configured to measure a pulse rate of the user based on the pulse wave signal; an interval calculation unit configured to calculate a data group of pulse wave intervals based on the pulse wave signal; a clustering unit configured to cluster the data group of the pulse wave intervals into one or more clusters by using a threshold; and a determination unit configured to determine, based on an index value indicating a magnitude of variation of the data group belonging to the cluster, whether atrial fibrillation occurs in the user. The clustering unit sets the threshold based on the pulse rate.

### CROSS-REFERENCE TO RELATED APPLICATIONS

This application is the U.S. national stage application filed pursuant to 35 U.S.C. 365(c) and 120 as a continuation of International Patent Application No. PCT//P2023/028436, filed Aug. 3, 2023, which application claims priority to Japanese Patent Application No. 2022-197343, filed Dec. 9, 2022, which applications are incorporated herein by reference in their entireties.

### TECHNICAL FIELD

The present disclosure relates to a sphygmomanometer and, in particular, to a sphygmomanometer having a function to determine atrial fibrillation.

### BACKGROUND ART

Prompt recognition of atrial fibrillation that causes a heart disease has been desired. A conventional technology for estimating atrial fibrillation from pulse wave information acquired by a sphygmomanometer has been proposed. Specifically, multiple blood pressure measurements are performed in one measurement opportunity using the sphygmomanometer, and thus pulse wave intervals that are intervals of a pulse wave signal acquired in each of the blood pressure measurements are acquired, and atrial fibrillation is detected based on the pulse wave intervals.

For example, a blood pressure measurement device that can indicate the presence or absence of atrial fibrillation is disclosed in U.S. Unexamined Patent Application Publication No. 2016/0228017 (Patent Document 1).

### CITATION LIST

### Patent Literature

Patent Document 1: US 2016/0228017 A

### SUMMARY OF INVENTION

### Technical Problem

In the device disclosed in Patent Document 1, in order to determine the presence or absence of atrial fibrillation, it is necessary to repeat a sequence defined by a predetermined pulse rate or the like, continuously multiple times (for example, three times) in one measurement opportunity. Therefore, the time required for the measurement becomes long, and the measurement site is compressed by a cuff to give a feeling of restraint to a user, which may place a burden on the user.

The present disclosure is intended in some respects to provide a sphygmomanometer that can accurately determine the presence or absence of atrial fibrillation while reducing burdens on a user during blood pressure measurement.

### Solution to Problem

In an example of the present disclosure, a sphygmomanometer, includes: a blood pressure measurement unit configured to measure blood pressure of a user based on a pulse wave signal superimposed on a cuff pressure signal detected in a process of increasing or decreasing cuff pressure that indicates internal pressure of a cuff attached to a target measurement site of the user; a pulse rate measurement unit configured to measure a pulse rate of the user based on the pulse wave signal; an interval calculation unit configured to calculate a data group of pulse wave intervals based on the pulse wave signal; a clustering unit configured to cluster the data group of the pulse wave intervals into one or more clusters by using a threshold; and a determination unit configured to determine, based on an index value indicating a magnitude of variation of the data group belonging to the cluster, whether atrial fibrillation occurs in the user. The clustering unit sets the threshold based on the pulse rate.

According to the configuration above, the presence or absence of atrial fibrillation can be accurately determined while reducing burdens on a user during blood pressure measurement.

In another example of the present disclosure, the clustering unit increases the threshold the lower the pulse rate.

According to the configuration above, since the threshold is appropriately set according to the pulse rate, an appropriate atrial fibrillation determination can be performed for each user.

In another example of the present disclosure, the threshold is set to be equal to or less than an average value of the data group of the pulse wave intervals. According to the configuration above, the possibility that an arrhythmia (for example, extrasystole) other than atrial fibrillation is erroneously determined to be atrial fibrillation can be reduced.

In another example of the present disclosure, when the data group of the pulse wave intervals is clustered into one cluster, the determination unit determines that atrial fibrillation occurs in the user, if a first index value indicating a magnitude of variation of the data group belonging to the one cluster is a predetermined value or more.

According to the configuration above, an atrial re-determination can be performed more accurately.

In another example of the present disclosure, when the data group of the pulse wave intervals is clustered into a plurality of clusters, the determination unit determines that atrial fibrillation occurs in the user, if a second index value indicating a magnitude of variation of the data group belonging to the plurality of clusters is a predetermined value or more.

According to the configuration above, an atrial re-determination can be performed more accurately.

In another example of the present disclosure, when the second index value is less than the predetermined value, the determination unit determines that an arrhythmia other than atrial fibrillation occurs in the user.

According to the configuration above, the presence or absence of occurrence of an arrhythmia other than atrial fibrillation can also be determined.

### Advantageous Effects of Invention

According to the present disclosure, the presence or absence of atrial fibrillation can be accurately determined while reducing burdens on a user during blood pressure measurement.

### BRIEF DESCRIPTION OF DRAWINGS

FIG. 1 is a diagram illustrating a sphygmomanometer according to a present embodiment.

FIG. 2 is a block diagram illustrating an example of a hardware configuration of the sphygmomanometer.

FIG. 3 is a block diagram illustrating a functional configuration of the sphygmomanometer.

FIG. 4(a), FIG. 4(b), and FIG. 4(c) are diagrams illustrating an example of a data group of pulse wave intervals.

FIG. 5 is a diagram for describing a clustering method.

FIG. 6 is a diagram showing the clustering results.

FIG. 7(a) and FIG. 7(b) are diagrams for describing a method of setting a threshold.

FIG. 8 is a diagram for describing an upper limit value of the threshold.

FIG. 9 is a flowchart for describing a processing procedure executed by the sphygmomanometer.

FIG. 10 is a flowchart illustrating an example of a blood pressure measurement process by the sphygmomanometer.

FIG. 11 is a flowchart illustrating another example of the blood pressure measurement process by the sphygmomanometer.

### DESCRIPTION OF EMBODIMENTS

Embodiments of the present invention will be described below with reference to the drawings. In the following description, the same components are denoted by the same reference numerals. Names and functions thereof are also the same. Thus, the detailed description of such components is not repeated.

### Application Example

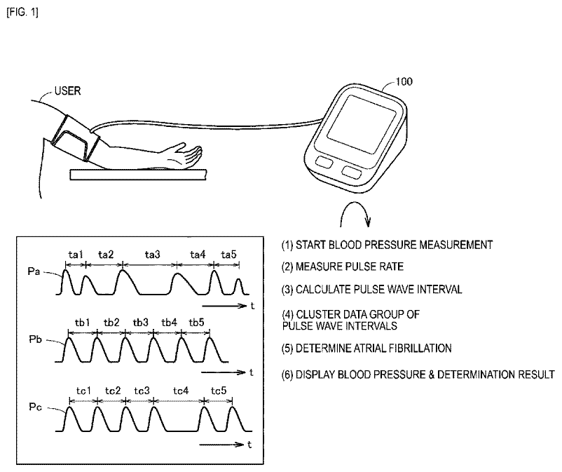

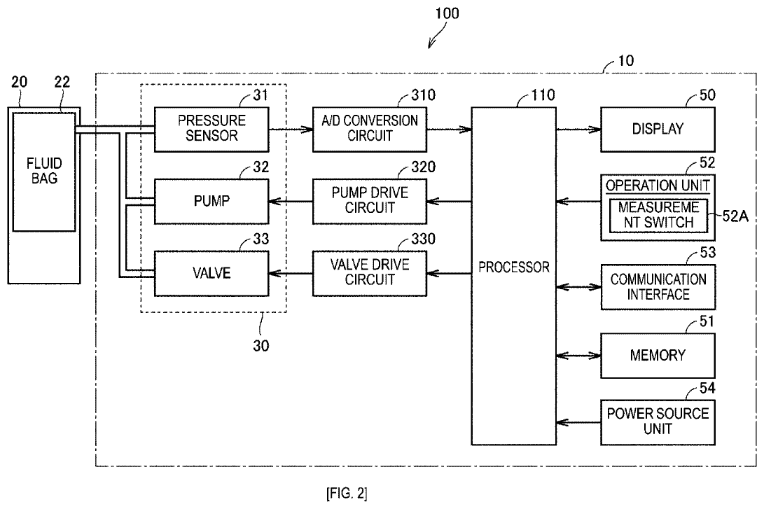

An application example of the present invention will be described with reference to FIG. 1. FIG. 1 is a diagram illustrating a sphygmomanometer 100 according to the present embodiment.

Referring to FIG. 1, the sphygmomanometer 100 is an upper arm type sphygmomanometer configured to measure blood pressure of a subject who is a user. The sphygmomanometer 100 includes a main body and a cuff (an arm band) as main components. The sphygmomanometer 100 may be a wrist type sphygmomanometer in which a main body and a cuff (an arm band) are integrated. Hereinafter, the processing content will be described with reference to FIG. 1.

In FIG. 1, a scene in which a user measures his/her blood pressure by using the sphygmomanometer 100 is assumed. The sphygmomanometer 100 starts blood pressure measurement in accordance with a blood pressure measurement instruction of the user (corresponding to (1) of FIG. 1). Specifically, the sphygmomanometer 100 extracts a pulse wave signal (fluctuation component) superimposed on a cuff pressure signal detected in a process of increasing or decreasing cuff pressure that indicates internal pressure of a cuff attached on a target measurement site (for example, an arm) of the user, and calculates a blood pressure value based on the pulse wave signal in accordance with an oscillometric method. For example, the sphygmomanometer 100 performs the blood pressure measurement by using a pressurization measurement method of measuring blood pressure in a pressurization process of the cuff pressure or a depressurization measurement method of measuring blood pressure in a depressurization process after the pressurization process of the cuff pressure.

The sphygmomanometer 100 measures (counts) a pulse rate (corresponding to (2) of FIG. 1) and calculates a data group of pulse wave intervals (corresponding to (3) of FIG. 1) based on a pulse wave signal obtained at the time of the blood pressure measurement (obtained in the pressurization process in the case of the pressurization measurement method, obtained in the depressurization process in the case of the depressurization measurement method). Typically, the pulse wave interval is a peak-to-peak interval of the pulse wave (or a bottom-to-bottom interval corresponding to the pulse wave).

For example, when a pulse wave signal Pa in FIG. 1 is obtained, a data group of pulse wave intervals ta1 to ta5 is calculated. Similarly, when a pulse wave signal Pb is obtained, a data group of pulse wave intervals tb1 to tb5 is calculated, and when a pulse wave signal Pc is obtained, a data group of pulse wave intervals tc1 to tc5 is calculated.

The pulse wave signal Pa is an example of a pulse wave signal indicating atrial fibrillation. The pulse wave intervals ta1 to ta5 in the pulse wave signal Pa vary irregularly in whole, and the pulse wave randomly occurs. The pulse wave signal Pb is an example of a pulse wave signal indicating normal sinus rhythm. The pulse wave intervals tb1 to tb5 in the pulse wave signal Pb are substantially the same, and the pulse wave regularly occurs. The pulse wave signal Pc is an example of a pulse wave signal indicating an arrhythmia (for example, extrasystole) other than atrial fibrillation. The pulse wave intervals tc1 to tc3 and tc5 in the pulse wave signal Pc are substantially the same; however, only the pulse wave interval tc4 is different in magnitude and the pulse wave is partially missing therein.

In the present embodiment, with a focus on the fact that pulse wave intervals in a pulse wave signal indicating atrial fibrillation vary irregularly in whole, the presence or absence of atrial fibrillation is determined by using an index value also referred to as clustered standard deviation (Cstd) that is an example of an index value indicating the magnitude of variation (hereinafter, also referred to as a “variation index value”).

The sphygmomanometer 100 generates one or more clusters by clustering the data group of the calculated pulse wave intervals with use of a threshold Th (corresponding to (4) of FIG. 1). For example, the sphygmomanometer 100 clusters the data group of the pulse wave intervals by comparing difference between each pulse wave interval included in the data group of the pulse wave intervals with the threshold Th.

For example, the data group of the pulse wave intervals ta1 to ta5 is classified into one cluster having a large variation between pieces of data. For the data group of the pulse wave intervals tb1 to tb5, one cluster having a small variation between pieces of data is generated. The data group of the pulse wave intervals tc1 to tc5 is classified into a cluster to which the pulse wave intervals tc1 to tc3 and tc5 belong and which has a small variation between pieces of data and into a cluster to which the pulse wave interval tc4 belongs.

The sphygmomanometer 100 determines the presence or absence of atrial fibrillation based on the variation index value of the data group belonging to the cluster (corresponding to (5) of FIG. 1). The data group of the pulse wave intervals ta1 to ta5 corresponding to atrial fibrillation has a large variation between pieces of data, and thus the variation index value of the data group is large. Meanwhile, the data group of the pulse wave intervals tb1 to tb5 corresponding to the normal sinus rhythm and the data group of the pulse wave intervals tc1 to tc5 corresponding to extrasystole have a small variation between pieces of data belonging to the cluster, and thus the variation index value of the data group is small. By using this, the sphygmomanometer 100 determines that atrial fibrillation has occurred when the variation index value of the data group belonging to the cluster is a predetermined value or more.

Then, the sphygmomanometer 100 displays the measured blood pressure value and the determination result of atrial fibrillation on a display (corresponding to (6) of FIG. 1).

According to the application example described above, in one measurement opportunity, blood pressure measurement and atrial fibrillation determination are performed at the same time and it is not necessary to perform blood pressure measurement multiple times for atrial fibrillation determination. As a result, in the blood pressure measurement, both the blood pressure measurement and atrial fibrillation determination can be performed while burdens on a user such as multiple and repeated compressions on the measurement site of the user and prolongation of blood pressure measurement time can be reduced. In addition, atrial fibrillation and an arrhythmia other than atrial fibrillation can be distinguished from each other by using the variation index value (for example, Cstd); therefore, the accuracy of atrial fibrillation determination can be improved.

### Configuration Example

### Hardware Configuration

FIG. 2 is a block diagram illustrating an example of a hardware configuration of the sphygmomanometer 100. Referring to FIG. 2, the sphygmomanometer 100 includes a main body 10 and a cuff 20 as main components. A fluid bag 22 is interiorly contained in the cuff 20. The main body 10 includes a processor 110, an air-system component 30 for blood pressure measurement, an A/D conversion circuit 310, a pump drive circuit 320, a valve drive circuit 330, a display 50, a memory 51, an operation unit 52, a communication interface 53, and a power source unit 54.

The processor 110 is an arithmetic processing unit such as a central processing unit (CPU) or a multi processing unit (MPU). The processor 110 reads and executes a program or recording medium stored in the memory 51 and thereby implements each processing (step) of the sphygmomanometer 100 described below. For example, the processor 110 performs control of driving a pump 32 and a valve 33 in accordance with an operation signal from the operation unit 52. In addition, the processor 110 calculates a blood pressure value by using an algorithm for blood pressure calculation according to the oscillometric method and displays the blood pressure value on the display 50.

The memory 51 is achieved by a random access memory (RAM), a read-only memory (ROM), a flash memory, or the like. The memory 51 stores a program or recording medium for controlling the sphygmomanometer 100, data used for controlling the sphygmomanometer 100, setting data for setting various functions of the sphygmomanometer 100, data of measurement results of blood pressure values, pulse rates, pulse wave intervals, and the like. In addition, the memory 51 is used as a working memory or the like when executing a program.

The air-system component 30 supplies or discharges air through an air line to or from the fluid bag 22 interiorly contained in the cuff 20. The air-system component 30 includes a pressure sensor 31 for detecting pressure inside the fluid bag 22, and the pump 32 and the valve 33 that serve as an expanding/contracting mechanism section for expanding/contracting the fluid bag 22.

The pressure sensor 31 detects the pressure (cuff pressure) inside the fluid bag 22 and outputs a signal (cuff pressure signal) corresponding to the detected pressure to the A/D conversion circuit 310. The pressure sensor 31 is, for example, a piezoresistive pressure sensor connected via the air line to the pump 32, the valve 33, and the fluid bag 22 interiorly contained in the cuff 20. The pump 32 supplies air as a fluid to the fluid bag 22 through the air line in order to increase the cuff pressure. The valve 33 is opened and closed to control the cuff pressure by discharging air inside the fluid bag 22 through the air line or filling air into the fluid bag 22.

The A/D conversion circuit 310 converts an output value of the pressure sensor 31 (e.g., a voltage value corresponding to a change in electric resistance due to a piezoresistive effect) from an analog signal to a digital signal and outputs the converted signal to the processor 110. The processor 110 acquires a signal representing the cuff pressure in accordance with the output value of the A/D conversion circuit 310. The pump drive circuit 320 controls driving of the pump 32 based on a control signal provided from the processor 110. The valve drive circuit 330 controls opening and closing of the valve 33 based on a control signal provided from the processor 110.

The processor 110 executes blood pressure measurement by the pressurization measurement method of measuring blood pressure of the user based on a pulse wave signal in a pressurization process of increasing the cuff pressure or the depressurization measurement method of measuring blood pressure of the user based on a pulse wave signal in a depressurization process of decreasing the cuff pressure after the pressurization process of increasing the cuff pressure to pressure greater than specified pressure (e.g., “estimated systolic blood pressure” described below).

For example, at the time of measurement by the depressurization measurement method, the following operation is generally performed. The cuff 20 is wound around a target measurement site (wrist, arm, etc.) of the user in advance, and at the time of measurement, the pump 32 and the valve 33 are controlled to increase the cuff pressure to be higher than the estimated systolic blood pressure and then gradually decrease the cuff pressure. In the process of depressurization, the cuff pressure is detected by the pressure sensor 31, and variation of the arterial volume occurring in the artery of the target measurement site is taken out as a pulse wave signal. The systolic blood pressure (maximal blood pressure) and the diastolic blood pressure (minimal blood pressure) are calculated based on the change (mainly rise and fall) in the amplitude of the pulse wave signal accompanying the change in the cuff pressure at that time.

The display 50 displays various kinds of information including results of the blood pressure measurement, atrial fibrillation measurement, and the like based on a control signal from the processor 110. The communication interface 53 exchanges various kinds of information with an external device. The power source unit 54 supplies power to the processor 110 and each piece of hardware.

The operation unit 52 inputs an operation signal corresponding to an instruction from the user to the processor 110. The operation unit 52 includes, for example, a measurement switch 52A for receiving a blood pressure measurement start instruction from the user.

### Functional Configuration

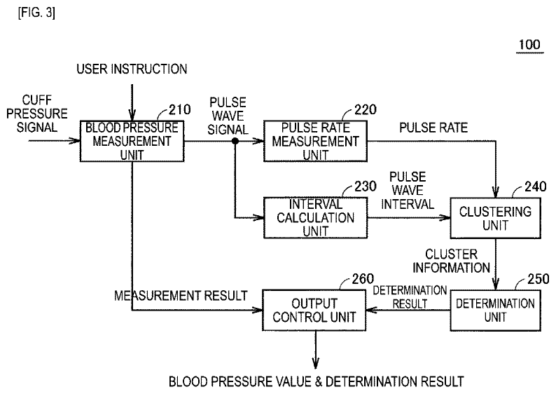

FIG. 3 is a block diagram illustrating a functional configuration of the sphygmomanometer 100. Referring to FIG. 3, the sphygmomanometer 100 includes, as a main functional configuration, a blood pressure measurement unit 210, a pulse rate measurement unit 220, an interval calculation unit 230, a clustering unit 240, a determination unit 250, and an output control unit 260. Each of these functions is realized, for example, by the processor 110 of the sphygmomanometer 100 executing a program stored in the memory 51. Note that some or all of these functions may be configured to be realized by hardware.

The blood pressure measurement unit 210 controls the cuff pressure in accordance with a measurement start instruction from the user via the operation unit 52 (for example, by pushing down the measurement switch 52A). Specifically, the blood pressure measurement unit 210 performs control of driving the pump 32 via the pump drive circuit 320 and driving the valve 33 via the valve drive circuit 330. The valve 33 is opened and closed to discharge or fill air from or into the fluid bag 22 and control the cuff pressure.

The blood pressure measurement unit 210 receives a cuff pressure signal detected by the pressure sensor 31 and extracts a pulse wave signal representing the pulse wave of the target measurement site superimposed on the cuff pressure signal. In other words, the blood pressure measurement unit 210 detects, from the cuff pressure signal, a pulse wave that is a pressure component superimposed on the cuff pressure signal in synchronization with the user's heartbeat.

The blood pressure measurement unit 210 measures blood pressure of the user based on the pulse wave signal superimposed on the cuff pressure signal detected in the process of increasing or decreasing the cuff pressure. Specifically, the blood pressure measurement unit 210 measures the blood pressure of the user by the pressurization measurement method or the depressurization measurement method in accordance with the oscillometric method. For example, in a case where the depressurization measurement method of detecting a pulse wave during depressurization of the fluid bag 22 is employed, the blood pressure measurement unit 210 calculates systolic blood pressure based on the cuff pressure when the amplitude of the pulse wave signal rapidly increases (at the time of rising) and diastolic blood pressure based on the cuff pressure when the amplitude of the pulse wave signal rapidly decreases (at the time of falling). Note that the blood pressure measurement unit 210 may employ a so-called pressurization measurement method of detecting a pulse wave when the fluid bag 22 is pressurized.

The pulse rate measurement unit 220 measures a pulse rate N of the user based on the pulse wave signal obtained at the time of the blood pressure measurement by the blood pressure measurement unit 210. Specifically, when the blood pressure measurement is performed by the pressurization measurement method, the pulse rate measurement unit 220 measures the pulse rate N based on the pulse wave signal in the pressurization process of the cuff pressure. When the blood pressure measurement is performed by the depressurization measurement method, the pulse rate measurement unit 220 measures the pulse rate N based on the pulse wave signal in the depressurization process of the cuff pressure.

The interval calculation unit 230 calculates a data group of pulse wave intervals based on the pulse wave signal. Specifically, when the blood pressure measurement is performed by the pressurization measurement method, the interval calculation unit 230 calculates a data group of pulse wave intervals indicated by the pulse wave signal based on the pulse wave signal in the pressurization process of the cuff pressure. When the blood pressure measurement is performed by the depressurization measurement method, the interval calculation unit 230 calculates a data group of pulse wave intervals indicated by the pulse wave signal based on the pulse wave signal in the depressurization process of the cuff pressure. For example, when a pulse wave signal Pa in FIG. 1 is obtained, a data group of pulse wave intervals is the pulse wave intervals ta1 to ta5.

The clustering unit 240 uses the threshold Th to cluster the data group of the pulse wave intervals into one or more clusters. Specifically, the clustering unit 240 sorts the data group of the pulse wave intervals in ascending order or descending order, and compares difference between adjacent pieces of data (the adjacent pulse wave intervals) with the threshold Th to cluster the data group of the pulse wave intervals, thereby generating one or more clusters.

In addition, the clustering unit 240 sets the threshold Th based on the pulse rate N. Specifically, the clustering unit 240 increases the threshold Th the lower the pulse rate N. Note that the threshold Th is set to be equal to or less than an average value of the data group of the pulse wave intervals. Details of a method of clustering the data group of the pulse wave intervals will be described below.

The determination unit 250 receives an input of cluster information by the clustering unit 240. The cluster information includes the number of clusters, information indicating the data group belonging to each cluster, and the like. The determination unit 250 calculates a variation index value of the data group belonging to the cluster based on the cluster information, and determines based on the variation index value whether atrial fibrillation has occurred in the user.

In an aspect, when the data group of the pulse wave intervals is clustered into one cluster, the determination unit 250 determines that atrial fibrillation has occurred in the user if the variation index value of the data group belonging to the one cluster is the predetermined value or more.

When the number of clusters is one cluster, any one of a standard deviation, a variance, a mean absolute deviation, and a median absolute deviation is used as the variation index value. Specifically, the determination unit 250 calculates any one of a standard deviation, a variance, a mean absolute deviation, and a median absolute deviation of the data group belonging to the one cluster as the variation index value.

In another aspect, when the data group of the pulse wave intervals is clustered into a plurality of clusters, the determination unit 250 determines that atrial fibrillation has occurred in the user if the variation index value of the data group belonging to the plurality of clusters is the predetermined value or more. Additionally, when the variation index value is less than the predetermined value, the determination unit 250 determines that an arrhythmia other than atrial fibrillation has occurred in the user.

When the number of clusters is a plurality of clusters, any one of clustered standard deviation (Cstd), “clustered variance (also referred to as “CVar” for convenience)”, “clustered absolute deviation (also referred to as “CAD” for convenience)”, an average value of standard deviations, an average value of variances, an average value of average absolute deviations, and an average value of median absolute deviations is used as the variation index value. Specifically, the determination unit 250 calculates any one of Cstd, CVar, CAD, an average value of standard deviations, an average value of variances, an average value of average absolute deviations, and an average value of median absolute deviations as the variation index value of the data group belonging to the plurality of clusters.

It is assumed that the Cstd is used as the variation index value. In this case, the determination unit 250 calculates the sum of squared deviations of the data group belonging to each cluster, and calculates a square root of a value obtained by dividing a total value of the sum of squared deviations by the total number of pieces of data of the pulse wave intervals as the Cstd. More specifically, the total number of pieces of data of the pulse wave intervals is N, the number of clusters is m, an average value of the data group in the cluster is xₐᵥ, the number of pieces of data of the data group is n, a data value included in the data group is xᵢ (where i=1 to n), and the sum of squared deviations of the data group is Sₖ (where k=1 to m). In this case, the Cstd is calculated by using the following equations (1) and (2).

$$\begin{matrix}Equation 1 & \\ {S}_{k} = {\sum }_{i = 1}^{n} {( {x}_{av} - {x}_{i} )}^{2} & ( 1 )\end{matrix}$$

$$Cstd = \sqrt{\frac{1}{N} {\sum }_{k = 1}^{m} {S}_{k}} \qquad ( 2 )$$

It is assumed that the CVar is used as the variation index value. In this case, the determination unit 250 calculates the sum of squared deviations of the data group belonging to each cluster, and calculates a value obtained by dividing a total value of the sum of squared deviations by the total number of pieces of data of the pulse wave intervals as the CVar. More specifically, the CVar is calculated by using the equation (1) and the following equation (3).

$$\begin{matrix}Equation 2 & \\ Cvar = \frac{1}{N} {\sum }_{k = 1}^{m} {AS}_{k} & ( 3 )\end{matrix}$$

It is assumed that the CAD is used as the variation index value. In this case, the determination unit 250 calculates the sum of absolute deviations of the data group belonging to each cluster, and calculates a value obtained by dividing a total value of the sums of absolute deviation by the total number of pieces of data of the pulse wave intervals as the CAD. More specifically, when the sum of absolute deviations of the data group in the cluster is Tₖ (where k=1 to m), the CAD is calculated by using the following equations (4) and (5). Note that other variables (for example, xₐᵥ and the like) are the same as those used in the equation (1).

$$\begin{matrix}Equation 3 & \\ {T}_{k} = {\sum }_{i = 1}^{n} \vert {x}_{av} - {x}_{i} \vert & ( 4 )\end{matrix}$$

$$CAD = \frac{1}{N} {\sum }_{k = 1}^{m} {T}_{k} \qquad ( 5 )$$

The average value of the standard deviations, the average value of the variances, the average value of the average absolute deviations, and the average value of the median absolute deviations are used as the variation index values are calculated as follows. Specifically, the determination unit 250 calculates the standard deviation of the data group belonging to each cluster, and calculates a value obtained by dividing a total value of the standard deviations by the number of clusters m as the “average value of the standard deviations”. The determination unit 250 calculates the variance of the data group belonging to each cluster, and calculates a value obtained by dividing a total value of the variances by the number of clusters m as the “average value of the variances”.

The determination unit 250 calculates the average absolute deviation (a value obtained by dividing the sum of absolute deviations by the number of pieces of data n) of the data group belonging to each cluster, and calculates a value obtained by dividing a total value of the average absolute deviations by the number of clusters m as the “average value of the average absolute deviations”. The determination unit 250 calculates the median absolute deviation of the data group belonging to each cluster, and calculates a value obtained by dividing a total value of the median absolute deviations by the number of clusters m as the “average value of the median absolute deviations”. Details of a method of determining atrial fibrillation will be described below.

The output control unit 260 displays the measurement result of the blood pressure measurement unit 210 (for example, systolic blood pressure and diastolic blood pressure values) and the determination result of the determination unit 250 (for example, the determination result of the presence or absence of occurrence of atrial fibrillation) on the display 50. Note that the output control unit 260 may transmit the measurement result and the determination result to an external device via the communication interface 53, or may be configured to output a voice via a speaker (not illustrated).

### Clustering and Determination of Atrial Fibrillation

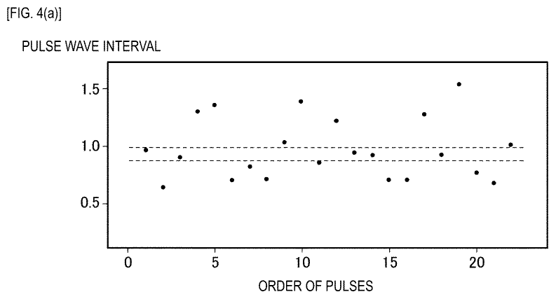

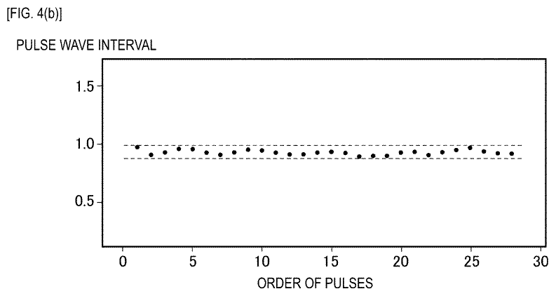

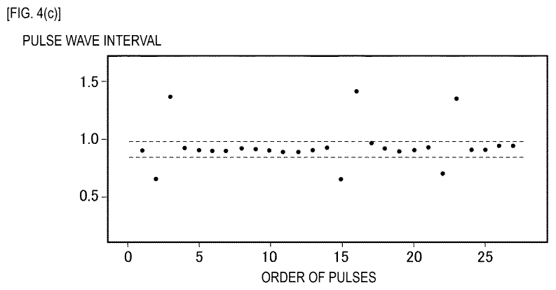

FIG. 4(a), FIG. 4(b), and FIG. 4(c) are diagrams illustrating an example of a data group of pulse wave intervals. Specifically, FIG. 4(a) is an example of a data group of pulse wave intervals in a pulse wave signal indicating atrial fibrillation. FIG. 4(b) is an example of a data group of pulse wave intervals in a pulse wave signal indicating normal sinus rhythm. FIG. 4(c) is an example of a data group of pulse wave intervals in a pulse wave signal indicating extrasystole. The vertical axis in each of FIG. 4(a) to FIG. 4(c) indicates a value obtained by standardizing the pulse wave intervals, and the horizontal axis indicates the order of the generated pulses.

The value indicated on the vertical axis in FIG. 4(a), FIG. 4(b), and FIG. 4(c) is a standardized value obtained by dividing a value of each of the pulse wave intervals included in the data group of the pulse wave intervals by an average value of the pulse wave intervals. Therefore, when a pulse wave interval T is equal to the average value, the value obtained by standardizing the pulse wave interval Tis “1”.

Referring to FIG. 4(a), the data group of the pulse wave intervals varies irregularly between about 0.6 and 1.5. This tendency is similar to that of the data group of the pulse wave intervals ta1 to ta5 in the pulse wave signal Pa in FIG. 1.

Referring to FIG. 4(b), no variation is present in the data group of the pulse wave intervals, and each piece of data is present near 1.0. This tendency is similar to that of the data group of the pulse wave intervals tb1 to tb5 in the pulse wave signal Pb in FIG. 1.

Referring to FIG. 4(c), in the data group of the pulse wave intervals, variations are present in a part of pieces of data. Specifically, although many pieces of data are present near 1.0, a part of pieces of data (for example, the 2nd, 3rd, 15th, 16th, 22nd, and 23rd pieces of data) have slightly different values. This tendency is similar to that of the data group of the pulse wave intervals tc1 to tc5 in the pulse wave signal Pc in FIG. 1.

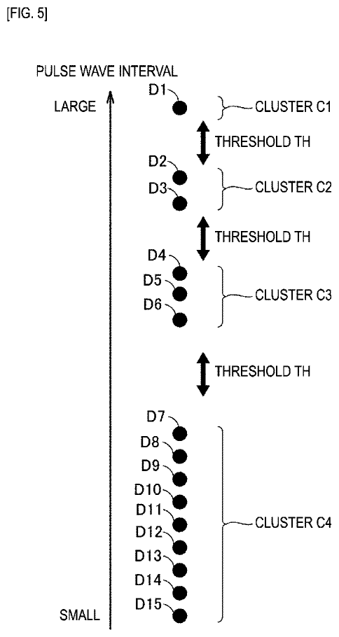

The sphygmomanometer 100 (the clustering unit 240) clusters the data group of the pulse wave intervals illustrated in FIG. 4(a), FIG. 4(b), and FIG. 4(c) to generate one or more clusters. First, the clustering method will be described with reference to FIG. 5.

FIG. 5 is a diagram for describing the clustering method. Referring to FIG. 5, it is assumed that data groups D1 to D15 are clustered. The data groups D1 to D15 are obtained by rearranging data groups of pulse wave intervals in a pulse wave signal in descending order. In other words, the data D1 is largest and the data D15 is smallest.

Clustering is executed by comparing difference between adjacent pieces of data with the threshold Th. For example, the sphygmomanometer 100 (e.g., the clustering unit 240) determines whether the difference between the data D1 and the data D2 subsequent to (adjacent to) the data D1 is the threshold Th or more. Since the difference is the threshold Th or more, the data D1 is classified into a cluster different from a cluster to which the data D2 belongs. In the example of FIG. 5, the data D1 belongs to a cluster C1.

Similarly, since the difference between the data D2 and the data D3 is less than the threshold Th, the clustering unit 240 classifies the data D2 and the data D3 into the same cluster. Subsequently, since the difference between the data D3 and the data D4 is the threshold Th or more, the clustering unit 240 classifies the data D3 into the cluster different from a cluster into which the data D4 belongs. Therefore, in the example of FIG. 5, the data D2 and the data D3 belong to a cluster C2.

By repeating the aforementioned processing, the data D1 belongs to the cluster C1, the data D2 and the data D3 belong to the cluster C2, the data D4 to the data D6 belong to a cluster C3, and the data D7 to the data D15 belong to a cluster C4. In the example of FIG. 5, the clustering unit 240 uses the threshold Th to cluster the data groups D1 to D15 of the pulse wave intervals into the four clusters C1 to C4.

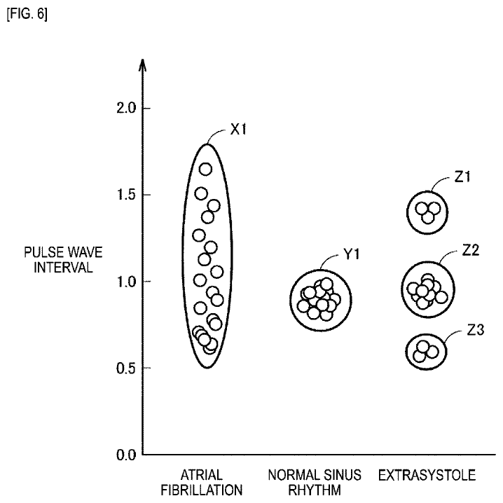

FIG. 6 is a diagram showing the clustering results. Specifically, FIG. 6 shows the results obtained by clustering each of the data groups of the pulse wave intervals illustrated in FIG. 4(a) to FIG. 4(c) with the use of the threshold Th. According to the clustering method described in FIG. 5, the data group of the pulse wave intervals related to atrial fibrillation illustrated in FIG. 4(a) is clustered into one cluster X1. The data group of the pulse wave intervals related to normal sinus rhythm illustrated in FIG. 4(b) is clustered into one cluster Y1. The data group of the pulse wave intervals related to extrasystole illustrated in FIG. 4(c) is clustered into three clusters Z1 to Z3.

Since the data group of the pulse wave intervals related to atrial fibrillation is clustered into the one cluster X1, any one of the four index values (i.e., standard deviation, variance, mean absolute deviation, and median absolute deviation) is used as a variation index value Sdx of the data group. As shown in FIG. 6, the data group belonging to the cluster X1 has a large variation, and thus the variation index value Sdx of the data group is large. Similarly, since the data group of the pulse wave intervals related to normal sinus rhythm is clustered into the one cluster Y1, any one of the four index values is used as a variation index value Sdy of the data group. Note that the same type of index value is used as the variation index values Sdx, Sdy. As shown in FIG. 6, the data group belonging to the cluster Y1 has a small variation, and thus the variation index value Sdy of the data group is small.

Since the data group of the pulse wave intervals related to extrasystole is clustered into the three clusters Z1 to Z3, any one of Cstd, CVar, CAD, an average value of standard deviations, an average value of variances, an average value of average absolute deviations, and an average value of median absolute deviations is used as a variation index value Sdz of the data group. As shown in FIG. 6, the data group belonging to each of the clusters Z1 to Z3 has a small variation. Therefore, the variation index value Sdz of the data group of pulse wave intervals corresponding to extrasystole is small.

Accordingly, when atrial fibrillation occurs, the variation index value calculated by the aforementioned method is large. Therefore, the sphygmomanometer 100 (the determination unit 250) calculates a variation index value of the data group belonging to the clustered cluster and, if the variation index value is larger than the predetermined value, determines that atrial fibrillation has occurred.

In addition, when the data group of the pulse wave intervals is clustered into a plurality of clusters, the sphygmomanometer 100 (the determination unit 250) determines that atrial fibrillation has occurred, if the variation index value of the data group belonging to the plurality of clusters is the predetermined value or more. This is because it is considered that although the plurality of clusters are generated, the data group belonging to each cluster has a large variation and the data group of the pulse wave intervals varies irregularly in whole (that is, the variation index value is large).

On the other hand, if the variation index value of the data group belonging to each of the plurality of clusters is less than the predetermined value, the sphygmomanometer 100 (the determination unit 250) determines that an arrhythmia (for example, extrasystole) other than atrial fibrillation has occurred.

Additionally, when the data group of the pulse wave intervals is clustered into one cluster and the variation index value of the data group belonging to the one cluster is less than the predetermined value, the sphygmomanometer 100 (the determination unit 250) may determine that the pulse of a user is normal (for example, normal sinus rhythm is indicated).

In order to appropriately determine the presence or absence of atrial fibrillation as described above, clustering needs to be performed with the use of the appropriate threshold Th. Hereinafter, a method of setting the threshold Th will be described.

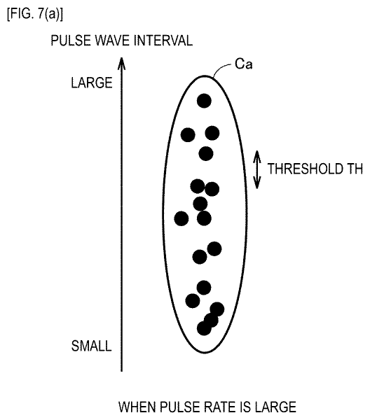

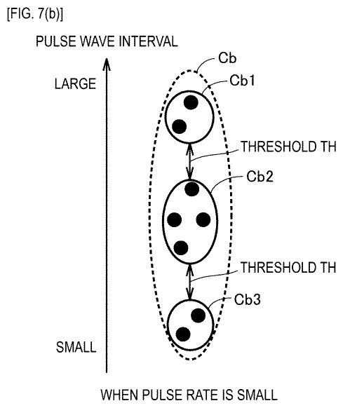

FIG. 7(a) and FIG. 7(b) are diagrams for describing the method of setting the threshold. FIG. 7(a) and FIG. 7(b) each show the result obtained by clustering the data group of the pulse wave intervals related to atrial fibrillation with the use of the threshold Th. FIG. 7(a) shows the clustering result when the pulse rate is large, and FIG. 7(b) shows the clustering result when the pulse rate is small.

In FIG. 7(a), one cluster Ca is generated, and the data group of the pulse wave intervals belonging to the cluster Ca has a large variation, and thus the variation index value (for example, standard deviation, variance, mean absolute deviation, or median absolute deviation) of the data group is large. Therefore, it is correctly determined that atrial fibrillation has occurred.

On the other hand, in FIG. 7(b), three clusters Cb1 to Cb3 are generated, and each of the data groups of the pulse wave intervals belonging respectively to the clusters Cb1 to Cb3 has a small variation. Thus, the variation index value (for example, Cstd, CVar, CAD, an average value of standard deviations, an average value of variances, an average value of mean absolute deviations, or an average value of median absolute deviations) of each of the data groups in a plurality of the clusters Cb1 to Cb3 is also small. Therefore, it is erroneously determined that an arrhythmia other than atrial fibrillation has occurred.

In order to prevent the erroneous determination as described above, the sphygmomanometer 100 (the clustering unit 240) changes the threshold Th according to the pulse rate. Specifically, the clustering unit 240 increases the threshold Th the lower the pulse rate. According to such a configuration, when the pulse rate is small as shown in FIG. 7(b), the threshold Th is large, and thus the clustering unit 240 generates one cluster Cb instead of generating the three clusters Cb1 to Cb3. Since the data group of the pulse wave intervals belonging to the cluster Cb varies greatly, the variation index value of the data group is large. Therefore, it is correctly determined that atrial fibrillation has occurred.

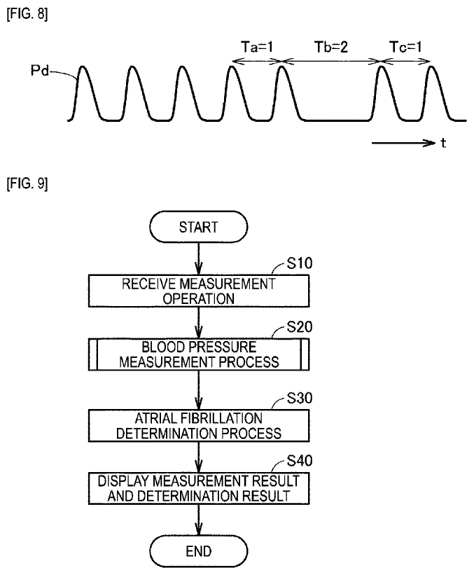

In addition, an upper limit value of the threshold Th can be considered as follows with the use of FIG. 8.

FIG. 8 is a diagram for describing the upper limit value of the threshold. Referring to FIG. 8, an example is illustrated in which one pulse is missing in a pulse wave signal Pd. As described above, when a pulse wave interval is equal to the average value of the data group of the pulse wave intervals, the value obtained by standardizing the pulse wave interval is “1”. In the example of FIG. 8, pulse wave intervals Ta, Tc are “1”. Next, when one pulse is missing, a pulse wave interval Tb locate between the pulses in front of and behind the missing pulse is “2”. Therefore, the difference between the pulse wave interval Tb and the pulse wave interval adjacent thereto is “1” (i.e., 2−1=1), which coincides with the average value of the data group of the pulse wave intervals.

The phenomenon of missing pulse is a phenomenon frequently observed in an arrhythmia (for example, extrasystole) other than atrial fibrillation. Therefore, when the threshold Th is set to “1” or more, the data group related to extrasystole is likely to be clustered as a single cluster instead of a plurality of clusters. In this case, an arrhythmia other than atrial fibrillation is likely to be erroneously determined to be “atrial fibrillation”. Consequently, the upper limit value of the threshold Th is set to the average value of the data group of the pulse wave intervals.

### Processing Procedure

FIG. 9 is a flowchart for describing a processing procedure executed by the sphygmomanometer 100. Referring to FIG. 9, at the start of such processing, the cuff 20 is attached to a target measurement site of a user.

Referring to FIG. 9, the processor 110 of the sphygmomanometer 100 receives, from the operation unit 21, an operation signal based on an operation of the measurement switch 52A by the user (step S10). The processor 110 starts a blood pressure measurement process (step S20) in response to the operation signal. In the blood pressure measurement process, a blood pressure value, a pulse rate N, and a data group of pulse wave intervals is calculated based on a pulse wave signal. Details of the blood pressure measurement process will be described below.

The processor 110 performs an atrial fibrillation determination process based on the pulse rate N and the data group of the pulse wave intervals (step S30). Specifically, the processor 110 sets the threshold Th used for clustering based on the pulse rate N, and clusters the data group of the pulse wave intervals into one or more clusters with the use of the threshold Th. Subsequently, the processor 110 determines whether atrial fibrillation has occurred based on a variation index value of the data group belonging to the cluster.

The processor 110 displays the blood pressure measurement result (for example, systolic blood pressure and diastolic blood pressure) and the atrial fibrillation determination result on the display 50 (step S40).

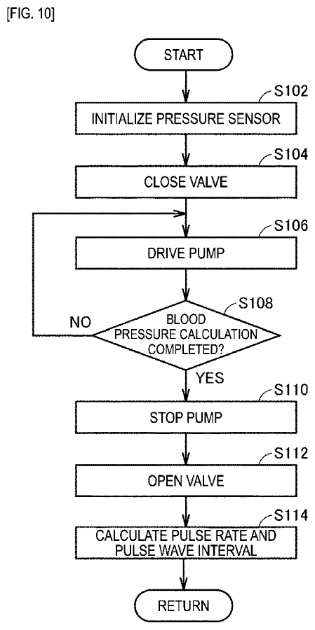

FIG. 10 is a flowchart illustrating an example of the blood pressure measurement process by the sphygmomanometer 100. The blood pressure measurement process illustrated in FIG. 10 (corresponding to step S20 of FIG. 9) is a process of measuring blood pressure by using the pressurization measurement method.

Referring to FIG. 10, the processor 110 of the sphygmomanometer 100 initializes the pressure sensor 31 (step S102). Specifically, the processor 110 initializes the processing memory area, and performs 0 mmHg adjustment (setting the atmospheric pressure to 0 mmHg) of the pressure sensor 31 in a state where the pump 32 is turned off (stopped) and the valve 33 is opened.

Next, the processor 110 closes the valve 33 via the valve drive circuit 330 (step S104), and turns on (activates) the pump 32 via the pump drive circuit 320 to start pressurization of the cuff 20 (the fluid bag 22) (step S106). At this time, the processor 110 controls the pressurization rate of the cuff pressure that is pressure inside the fluid bag 22 based on the output of the pressure sensor 31 while supplying air from the pump 32 through the air line to the fluid bag 22. This starts the pressurization process.

Next, the processor 110 extracts a pulse wave signal from the cuff pressure signal detected by the pressure sensor 31, attempts to calculate systolic blood pressure and diastolic blood pressure based on the pulse wave signal, and determines whether the blood pressure calculation has been completed (step S108).

If the blood pressure calculation has not been completed yet due to a lack of data (NO in step S108), the processor 110 repeats the processing of steps S106, S108 unless the cuff pressure reaches a predetermined upper pressure limit (e.g., 300 mmHg). If the blood pressure calculation has been completed (YES in step S108), the processor 110 performs control of stopping the pump 32 (i.e., stopping the pressurization process) (step S110), opening the valve 33 (step S112), and discharging the air inside the cuff 20.

The processor 110 calculates the pulse rate N and the data group of the pulse wave intervals based on the pulse wave signal obtained in the pressurization process (step S114). The processor 110 stores the calculated pulse rate N and data group of the pulse wave intervals in the memory 51.

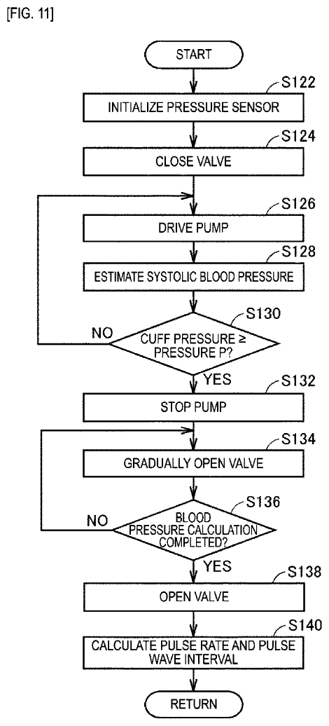

FIG. 11 is a flowchart illustrating another example of the blood pressure measurement process by the sphygmomanometer 100. The blood pressure measurement process illustrated in FIG. 11 (corresponding to step S20 of FIG. 9) is a process of measuring blood pressure by using the depressurization measurement method.

Referring to FIG. 11, since the processing of steps S122 to S126 is the same as the processing of steps S102 to S106 of FIG. 6, the detailed description thereof is not provided.

The processor 110 estimates systolic blood pressure based on a pulse wave signal obtained during pressurization (step S128). The processor 110 determines whether the cuff pressure has reached a pressure P or greater (step S130). Typically, the pressure P is set to a value higher than the estimated systolic blood pressure value by a fixed value (e.g., 40 mmHg).

When the cuff pressure is lower than the pressure P (NO in step S130), the processor 110 returns to step S126. If the cuff pressure is the pressure P or higher (YES in step S130), the processor 110 stops the pump 32 (step S132) and performs control of gradually opening the valve 33 (step S134). As a result, the pressurization process shifts to the depressurization process (i.e., the depressurization process is started), and the cuff pressure gradually decreases.

In the depressurization process, the processor 110 extracts a pulse wave signal from the cuff pressure signal detected by the pressure sensor 31, attempts to calculate systolic blood pressure and diastolic blood pressure based on the pulse wave signal, and determines whether the blood pressure calculation has been completed (step S136). If the blood pressure calculation has not been completed (NO in step S136), the processor 110 repeats the processing of steps S134 and S136. If the blood pressure calculation has been completed (YES in step S136), the processor 110 performs control of fully opening the valve 33 (step S138) and rapidly discharging the air inside the cuff 20.

The processor 110 measures the pulse rate N and the data group of the pulse wave intervals based on the pulse wave signal obtained in the depressurization process (step S140). The processor 110 stores the calculated pulse rate N and data group of the pulse wave intervals in the memory 51.

### Other Embodiments

(1) In the embodiments described above, a program may be provided that causes a computer to function and execute controls such as those described in the aforementioned flowcharts. Such a program can also be provided as a program product stored on a non-temporary computer-readable recording medium attached to a computer, such as a flexible disk, a compact disc read only memory (CD-ROM), a secondary storage device, a main storage device, and a memory card. Alternatively, a program may be provided by recording the program on a recording medium such as a hard disk built into a computer. A program may also be provided by download via a network.

(2) The configuration exemplified as the embodiments described above is an example of a configuration of the present invention, and the configuration can be combined with other known technology, and one part thereof may be omitted or modified without departing from the scope of the present invention. In addition, the processes and configurations described in other embodiments may be employed as appropriate in the embodiments described above.

### Supplementary Notes

As described above, the present embodiments include the following disclosures.

### Configuration 1

A sphygmomanometer (100) includes: a blood pressure measurement unit (210) configured to measure blood pressure of a user based on a pulse wave signal superimposed on a cuff pressure signal detected in a process of increasing or decreasing cuff pressure that indicates internal pressure of a cuff (20) attached to a target measurement site of the user; a pulse rate measurement unit (220) configured to measure a pulse rate of the user based on the pulse wave signal; an interval calculation unit (230) configured to calculate a data group of pulse wave intervals based on the pulse wave signal; a clustering unit (240) configured to cluster the data group of the pulse wave intervals into one or more clusters by using a threshold; and a determination unit (250) configured to determine, based on an index value indicating a magnitude of variation of the data group belonging to the cluster, whether atrial fibrillation occurs in the user. The clustering unit (240) sets the threshold based on the pulse rate.

### Configuration 2

The sphygmomanometer (100) according to Configuration 1, wherein the clustering unit (240) increases the threshold the lower the pulse rate.

### Configuration 3

The sphygmomanometer (100) according to Configuration 1 or 2, wherein the threshold is set to be equal to or less than an average value of the data group of the pulse wave intervals.

### Configuration 4

The sphygmomanometer (100) according to any one of Configurations 1 to 3, wherein when the data group of the pulse wave intervals is clustered into one cluster, the determination unit (250) determines that atrial fibrillation occurs in the user, if a first index value indicating a magnitude of variation of the data group belonging to the one cluster is a predetermined value or more.

### Configuration 5

The sphygmomanometer (100) according to any one of Configurations 1 to 4, wherein when the data group of the pulse wave intervals is clustered into a plurality of clusters, the determination unit (250) determines that atrial fibrillation occurs in the user, if a second index value indicating a magnitude of variation of the data group belonging to the plurality of clusters is a predetermined value or more.

### Configuration 6

The sphygmomanometer (100) according to Configuration 5, wherein when the second index value is less than the predetermined value, the determination unit (250) determines that an arrhythmia other than atrial fibrillation occurs in the user.

The embodiments disclosed herein are illustrative in all respects and are not intended as limitations. The scope of the present invention is indicated not by the descriptions above but by the claims and is intended to include all changes within the meaning and scope equal to the scope of the claims.

### REFERENCE NUMERALS LIST

10 Main body, 20 Cuff, 22 Fluid bag, 30 Air-system component, 31 Pressure sensor, 32 Pump, 33 Valve, 50 Display, 51 Memory, 52 Operation unit, 52A Measurement switch, 53 Communication interface, 54 Power source unit, 100 Sphygmomanometer, 110 Processor, 210 Blood pressure measurement unit, 220 Pulse rate measurement unit, 230 Interval calculation unit, 240 Clustering unit, 250 Determination unit, 260 Output control unit, 310 A/D conversion circuit, 320 Pump drive circuit, 330 Valve drive circuit

1. A sphygmomanometer comprising:
a blood pressure measurement unit configured to measure blood pressure of a user based on a pulse wave signal superimposed on a cuff pressure signal detected in a process of increasing or decreasing cuff pressure that indicates internal pressure of a cuff attached to a target measurement site of the user;
a pulse rate measurement unit configured to measure a pulse rate of the user based on the pulse wave signal;
an interval calculation unit configured to calculate a data group of pulse wave intervals based on the pulse wave signal;
a clustering unit configured to cluster the data group of the pulse wave intervals into one or more clusters by using a threshold; and
a determination unit configured to determine, based on an index value indicating a magnitude of variation of the data group belonging to the cluster, whether atrial fibrillation occurs in the user,
wherein
the clustering unit sets the threshold based on the pulse rate, and
when the data group of the pulse wave intervals is clustered into one cluster, the determination unit determines that atrial fibrillation occurs in the user, if a first index value indicating a magnitude of variation of the data group belonging to the one cluster is a predetermined value or more.

2. The sphygmomanometer according to claim 1, wherein
the clustering unit increases the threshold the lower the pulse rate.

3. The sphygmomanometer according to claim 1, wherein
the threshold is set to be equal to or less than an average value of the data group of the pulse wave intervals.

4. The sphygmomanometer according to claim 1, wherein
when the data group of the pulse wave intervals is clustered into one cluster, the determination unit determines that atrial fibrillation occurs in the user, if a first index value indicating a magnitude of variation of the data group belonging to the one cluster is a predetermined value or more.

5. The sphygmomanometer according to claim 1, wherein
when the second index value is less than the predetermined value, the determination unit determines that an arrhythmia other than atrial fibrillation occurs in the user.

6. A sphygmomanometer comprising:
a blood pressure measurement unit configured to measure blood pressure of a user based on a pulse wave signal superimposed on a cuff pressure signal detected in a process of increasing or decreasing cuff pressure that indicates internal pressure of a cuff attached to a target measurement site of the user;
a pulse rate measurement unit configured to measure a pulse rate of the user based on the pulse wave signal;
an interval calculation unit configured to calculate a data group of pulse wave intervals based on the pulse wave signal;
a clustering unit configured to sort the data group of the pulse wave intervals in ascending order or descending order and compare, in the sorted data group of the pulse wave intervals, difference between the adjacent two pulse wave intervals with a threshold to cluster the data group of the pulse wave intervals into one or more clusters; and
a determination unit configured to determine, based on an index value indicating a magnitude of variation of the data group belonging to the cluster, whether atrial fibrillation occurs in the user,
wherein
the clustering unit sets the threshold based on the pulse rate.
